(19) United States
(12) Patent Application Publication
(10) Pub. No.: US 2021/0055307 A1
(43) Pub. Date: Feb. 25, 2021

(54) METHOD FOR MONITORING DEVELOPMENT OF THE CENTRAL NERVOUS SYSTEM

(71) Applicant: Tammy Zietchick Movsas, Southfield, MI (US)

(72) Inventor: Tammy Zietchick Movsas, Southfield, MI (US)

(21) Appl. No.: 16/997,326

(22) Filed: Aug. 19, 2020

Related U.S. Application Data
(60) Provisional application No. 62/890,992, filed on Aug. 23, 2019.

(51) Int. Cl. G01N 33/68

(52) U.S. Cl. CPC G01N 33/6872; G01N 2800/7014; G01N 2800/38; G01N 2333/515

(57) ABSTRACT

The present invention relates generally to the field of medical diagnostics, and more particularly to a method for determining a vascular endothelial growth factor-A (“VEGF”) isoform concentration pattern of a biological sample. A biological material is obtained from a premature infant. A VEGF isoform concentration pattern of the biological material is determined using at least a first cytokine and a second cytokine. The VEGF isoform pattern is compared to a reference VEGF isoform concentration pattern that conveys a baseline concentration of at least a first cytokine and a second cytokine. A normal VEGF isoform concentration pattern is determined when the VEGF isoform concentration pattern is within a threshold percentage of the reference VEGF isoform concentration pattern. The VEGF isoform concentration pattern conveys concentration levels of at least the first cytokine and the second cytokine. The biological material may be a blood or a fecal sample obtained from a premature infant.

### CROSS-REFERENCE TO RELATED APPLICATIONS

This application claims priority to U.S. Provisional Application No. 60/778,829 filed Aug. 23, 2019. This application is hereby incorporated herein by reference.

### TECHNICAL FIELD

### Background

Infants born before 37 weeks of gestational age (hereafter “GA”) are considered premature and are at risk for complications. These complications can affect the development of the infant's central nervous system (hereinafter “CNS”). The CNS includes the brain, the retina and the spinal cord. Premature infants are at much higher risk of neurodevelopmental disorders. Examples of such include, but are not limited to, cerebral palsy, autism and retinopathy of prematurity. There is a direct relationship between the extent of prematurity and the risk of developing CNS complications. For example, at least 50% of children born at less than 30 weeks of GA typically display deficits in their visual, motor, cognitive and/or behavioral skills. Although head circumference is currently the standard screening tool for potential neuro-developmental impairment, it has limited utility. In the same vein, MRI imaging is of limited utility, since learning disabilities can still occur despite the presence of a normal MRI scan.

### BRIEF DESCRIPTION OF THE DRAWINGS

Some of the embodiments will be described in detail, with reference to the following figures, wherein like designations denote like members, wherein:

FIG. 1 depicts a method for determining a vascular endothelial growth factor-A (“VEGF”) isoform concentration pattern of a biological sample, according to some embodiments.

FIG. 2 depicts scatter plots of the VEGF165 levels versus the VEGF121 levels of infants using the method of FIG. 1.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below. Unless otherwise indicated, the drawings are intended to be read together with the specification and are to be considered a portion of the entire written description of this invention.

Infants born before 37 weeks of gestational age (hereafter “GA”) are considered premature and are at risk for complications. These complications can affect the development of the infant's central nervous system (hereinafter “CNS”). The CNS includes the brain, the retina and the spinal cord. Premature infants are at much higher risk of neurodevelopmental disorders. Examples of such include, but are not limited to, cerebral palsy, autism and retinopathy of prematurity. There is a direct relationship between the extent of prematurity and the risk of developing CNS complications. For example, at least 50% of children born at less than 30 weeks of GA typically display deficits in their visual, motor, cognitive and/or behavioral skills. Although head circumference is currently the standard screening tool for potential neuro-developmental impairment, it has limited utility. In the same vein, MRI imaging is of limited utility, since learning disabilities can still occur despite the presence of a normal MRI scan. Thus, for example, a practical or effective manner to monitor CNS development in infants is still lacking. The present disclosure includes many aspects and features. Moreover, while many aspects and features relate to, and are described in the context of medical diagnostic methods, embodiments of the present disclosure are not limited to use only in this context.

Embodiments of the present disclosure seek to provide methods for determining a vascular endothelial growth factor-A (hereinafter “VEGF”) isoform concentration pattern of a biological sample. VEGF isoforms are examples of cytokines. Cytokines are various substances, such as (but not limited to) interferon, interleukin, and growth factors, which have an effect on other cells. Certain embodiments of the instant disclosure seek to provide methods that can be used to distinguish between baseline and non-baseline VEGF isoform concentration patterns to ascertain the development of the human central nervous system. Embodiments disclosed herein seek to provide methods that ascertain the concentration of two or more VEGF isoforms concentration levels. Not to be limited by theory, VEGF is believed to be the primary driver of angiogenesis during neurodevelopment (i.e. brain and retina). Although many VEGF isoforms exist, VEGF165, VEGF121, and VEGF189 are the most prevalent human VEGF isoforms. Each VEGF isoform is believed to play a different role in angiogenesis. Other aspects of the instant disclosure seek to collects bodily fluid(s) (e.g., fecal matter, urine, blood, breast milk, other bodily fluids, or a combination of two or more thereof) and ascertain two or more VEGF isoform concentration levels therein at one or more time points post-birth.

Elevated (total) VEGF isoform concentration levels with concomitant aberrant angiogenesis are described in many neuro-related morbidities that include, but are not limited to periventricular leukomalacia, intraventricular hemorrhages, perinatal hypoxic-ischemic, brain injury, retinopathy of prematurity (hereinafter “ROP”) and autism. Such disorders are either co-morbidities or precursors of cerebral palsy, a neuro-developmental disorder whose prevalence is 50-fold higher in children born prematurely.

A time-dependent correlation of two or more VEGF isoforms in a premature infant is associated with normal (i.e. desired, within a statistically significant percentage of baseline, or similar) CNS development (i.e. an absence of a neuro-morbidity). An altered time-dependent correlation of two or more VEGF isoforms in a premature infant born of the same GA) is associated with abnormal CNS development. (i.e. the development or pending development of a neuro-morbidity. Retinopathy of prematurity (hereinafter “ROP”) is an example of a neuro-morbidity. ROP primarily develops in premature infants that are born at less than 28 weeks of GA. Not to be limited by theory, a time-dependent correlation of VEGF165/VEGF121 is associated with normal retinal development (hereinafter “no-ROP”) in premature infants (who are born at <28 weeks of GA). An altered time-dependent correlation of the two isoforms is associated with the development of ROP in premature infants (with matched gestational ages to the no-ROP infants).

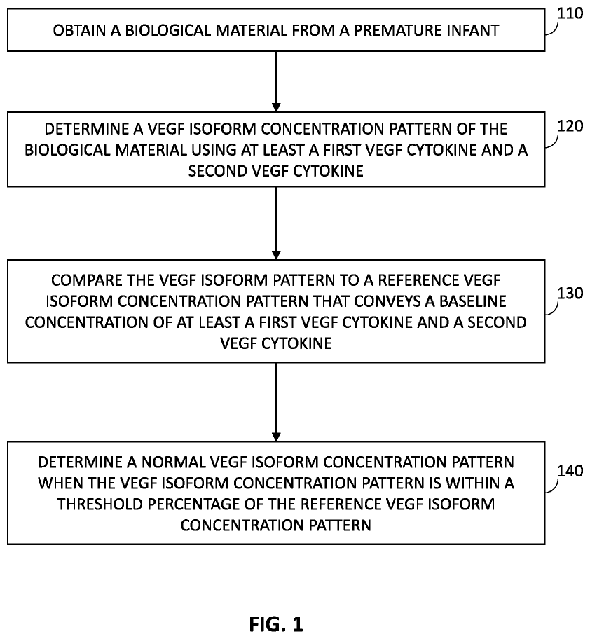

FIG. 1 depicts a method for determining a vascular endothelial growth factor-A (“VEGF”) isoform concentration pattern of a biological sample, according to some embodiments. At Step 110, biological material is obtained from a premature infant. For example, the premature infant can have a gestational age of greater than 24 weeks and up to 28 weeks (i.e. 24 wks.<GA≤28 wks.). For example, obtaining the biological material can include obtaining a blood sample from the premature infant. The blood sample can be blotted on the filter paper and dried thereon. In certain embodiments, obtaining the biological material can include obtaining a fecal sample from the infant. A VEGF isoform concentration pattern of the biological material is determined using at least a first VEGF cytokine and a second VEGF cytokine (Step 120). In one embodiment, the first VEGF cytokine includes VEGF165 and the second VEGF cytokine includes VEGF121. In other embodiments, determining the vascular VEGF isoform concentration pattern of the biological material further uses a third VEGF cytokine (e.g., VEGF189). For example, the VEGF isoform concentration pattern is preferably determined using an enzyme-linked immunosorbent assay (“ELISA”) and/or electrochemiluminescence platform. Other types of assay platforms may also be used. In other embodiments, VEGF isoform mRNA expression levels are measured instead of cytokine protein levels.

The VEGF isoform concentration pattern conveys concentration levels of the first VEGF cytokine and the second VEGF cytokine. The VEGF isoform concentration pattern is preferably compared to a reference VEGF isoform pattern that conveys baseline (i.e. normal development of the CNS) baseline concentration levels of a first VEGF cytokine and a second VEGF cytokine (Step 130). For example, the reference VEGF isoform pattern can be obtained from data provided by the biological material from several premature infants with gestational ages that approximate the GA of the first premature infants. In preferred embodiments, the reference VEGF isoform concentration pattern is gender specific and/or GA-specific (discussed further below). The reference VEGF isoform concentration pattern is preferably formed using a statistically significant sampling of patients that each exhibit baseline concentration levels of at least the first VEGF cytokine (e.g., VEGF165) and the second VEGF cytokine (e.g., VEGF121). The gestational age of the infants should be approximate. One example of approximate would be within a gestational age range of 4 weeks. A baseline VEGF isoform concentration pattern is determined when the VEGF isoform concentration pattern is within a threshold percentage (e.g., 5% or other desired percentage that reflects a similarity there between) of the reference VEGF isoform pattern (Step 140).

In embodiments that utilize the third VEGF isoform, the reference VEGF isoform concentration pattern preferably conveys a baseline concentration of at least the first VEGF cytokine, the second VEGF cytokine, and a third VEGF cytokine. Here, the VEGF isoform concentration pattern conveys concentration levels of at least the first VEGF cytokine, the second VEGF cytokine, and the third VEGF cytokine. As an example, the third VEGF cytokine may be VEGF189.

### EXAMPLE

Pilot study was conducted utilizing neonatal dried blood spots (hereinafter “DBS”) derived from whole blood specimens were collected from newborns within the first week of birth To form the DBS, several droplets of blood from the heel of each newborn had been directly spotted onto filter paper, air dried, and stored at −20° C. This study was a case-control (∓ROP versus no ROP) study of 65 preterm infants with birthweights (hereinafter “BW”) less than 1,500 grams and a GA of greater than 24 weeks and less than or equal to 28 weeks. The study population was composed of 27 cases of non-proliferative retinopathy of prematurity (ROP) (15 boys and 12 girls) and 38 non-cases (19 boys and 19 girls). This study was performed with the use of pre-collected, deidentified DBS. ICD9 codes were utilized to identify the ROP cases.

VEGF165 and VEGF121 concentration levels of the samples were measured. DBS from the infants with GAs between 24-28 weeks were analyzed. Since non-cases were not matched to cases, the no-ROP group is referred to as the non-case group as opposed to a control group. To be sure, ICD-9 codes from the hospital records were used to identify cases (non-proliferative ROP) and non-cases (no-ROP). Three databases were linked to hospital records (including the ICD9 codes utilized) in order to identify cases and non-cases. In brief, non-proliferative ROP cases were defined as any ROP without documented retinal neovascularization on the hospital record. All DBS samples were de-identified before their release for use. Individual diagnostic codes were not provided; instead, each DBS was classified as either non-proliferative ROP or no-ROP.

Additional medical information about the study participants was not available due to the de-identification process. Turning now to the analysis of VEGF165. For each neonatal DBS, a 3 mm punch was prepared for VEGF165 assessment by soaking in 120 μl of PBS buffer containing 0.05% Tween-20 and 0.08% sodium azide for 4 hours in ice. The same step was repeated for VEGF121 assessment. Electrochemiluminescence (hereinafter “ECL”) assays were performed on the MSD U-PLEX platform to assess the VEGF165 values according to manufacturer's instruction (MSD Human VEGF Kit, Gaithersburg, Md., USA). The MSD human VEGF kit is optimized for the detection and quantitation of the VEGF165 isoform.

To begin, VEGF antibodies were cross linked with an appropriate crosslinker and the activated U-PLEX ELISA plate coated with crosslinked VEGF antibodies overnight at 4° C. Plates were thrice washed with PBS-T buffer (Phosphate Buffered Saline with 0.05% Tween-20). Each well received 25 μl of DBS supernatant and 25 μl diluent 43 (supplied with kit), plates were sealed then incubated for 2 hours at room temperature on an ELISA plate shaker at 6500 rpm. After washing, each well received 25 μl of detection antibody reagent and incubated for 2 hours at room temperature on the shaker. Plates were thrice washed again with PBS-T. Each well received 150 μl of 1× READ buffer prepared with distilled water (supplied with kit) and immediately read on SECTOR® Imager 6000. To express analyte concentrations per total mg protein of the DBS extract, total protein of the DBS extracts was measured using Micro BSA protein assay kit (Thermo Fisher™, Catalogue #23235).

Turning now to the analysis of VEGF121. A 3-mm punch from each neonatal DBS was prepared for V121 assessments by soaking the punch in PBS buffer as described above. VEGF121 was measured in DBS extracts using the Cloud Clone ELISA kit for VEGF 121 (Catalogue #: SEB851Hu, Cloud Clone Corp., Wuhan, China) according to manufacturer's instructions. Wells of the pre-coated ELISA plates each received 100 μl of the Standard and samples per well in replicate including a blank (e.g., the Standard Diluent). Plates were sealed and incubated for 1 hour at 37° C. Wells were decanted and blotted on paper towels to remove any samples. Each wells subsequently received 100 μl of Detection Reagent A. The plates were sealed and incubated for 1 hour at 37° C. Wells were each decanted and thrice washed with 350 μl of Wash Buffer while undergoing shaking for 1-2 minutes at 6500 rpm. Next, 100 μl of Detection Reagent B was added to each well, and the sealed plates were incubated for 30 min at 37° C.

Plates were washed as above and 90 μl of Substrate Solution was added to each well. Each plate was subsequently sealed and incubated for 20 min at 37° C. in the dark. Immediately following the incubation, 50 μl of Stop Solution was added to each well and mixed thoroughly. Plates were read at 450 nm. To express analyte concentrations per total mg protein of the DBS extract, total protein of the DBS extracts was measured using a Micro BSA protein assay kit (Thermo Fisher™, Catalogue #23235) according to manufactures instructions.

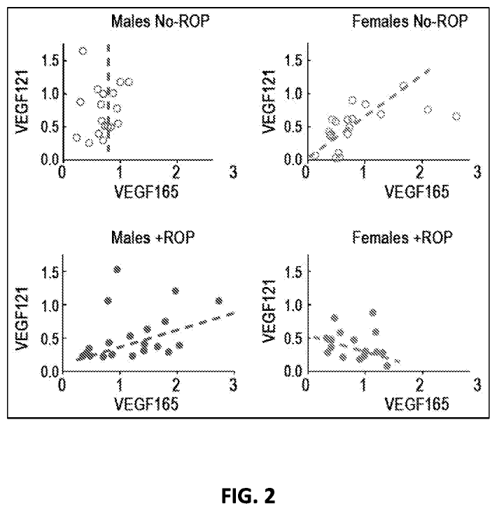

VEGF165 and VEGF121 were normalized to the assay-specific internal control. VEGF values for each DBS were normalized to an assay-specific internal control (normalized to 1 during each assay run). Thus, the VEGF values in this study represent relative values (without a unit of measure attached to them). Statistical analyses of the assay results were performed and the results were graphed. FIG. 2 depicts scatter plots of the VEGF165 levels versus the VEGF121 levels (with best-fit lines added for visualization clarity) of the infants in the pilot study using the method disclosed herein.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but should be defined only in accordance with the following claims appended hereto and their equivalents.

1. A method for determining a vascular endothelial growth factor-A (“VEGF”) isoform concentration pattern of a biological sample, comprising:
obtaining biological material from a premature infant;
determining a VEGF isoform concentration pattern of the biological material using at least a first VEGF cytokine and a second VEGF cytokine;
comparing the VEGF isoform concentration pattern to a reference VEGF isoform concentration pattern that conveys a baseline concentration of at least the first VEGF cytokine and the second VEGF cytokine;
determining a normal VEGF isoform concentration pattern when the VEGF isoform concentration pattern is within a threshold percentage of the reference VEGF isoform concentration pattern; and
wherein the VEGF isoform concentration pattern conveys concentration levels of at least the first VEGF cytokine and the second VEGF cytokine.

2. The method of claim 1, wherein obtaining the biological material comprises:
obtaining a blood sample from the premature infant;
blotting the blood sample on a filter paper; and
drying the blood sample on the filter paper.

3. The method of claim 1, wherein obtaining the biological material comprises obtaining a fecal sample from the premature infant.

4. The method of claim 1, wherein the first VEGF cytokine comprises VEGF165.

5. The method of claim 4, wherein the second VEGF cytokine comprises VEGF121.

6. The method of claim 5, wherein
determining the vascular VEGF isoform concentration pattern of the biological material further uses a third VEGF cytokine;
the reference VEGF isoform concentration pattern conveys a baseline concentration of at least the first VEGF cytokine, the second VEGF cytokine, and a third VEGF cytokine; and
the VEGF isoform concentration pattern conveys concentration levels of at least the first VEGF cytokine, the second VEGF cytokine, and the third VEGF cytokine.

7. The method of claim 6, wherein the third VEGF cytokine comprises VEGF189.

8. The method of claim 7, wherein the VEGF isoform pattern is determined using an enzyme-linked immunosorbent assay.

9. The method of claim 8, wherein
the reference VEGF isoform pattern is obtained from premature infants born within a 4-week gestational age range of the first infant.

10. The method of claim 9, wherein the reference VEGF isoform concentration pattern is gender specific.

11. The method of claim 10, further comprising forming the reference VEGF isoform pattern using a statistically significant sampling of patients that each exhibit two or more of:
a baseline concentration level of the first VEGF cytokine;
a baseline concentration level of the second VEGF cytokine; and
a baseline concentration level of the third VEGF cytokine.

12. A method for determining a vascular endothelial growth factor-A (“VEGF”) isoform concentration pattern of a biological sample, comprising:
obtaining biological material from a premature infant;
determining a vascular endothelial growth factor-A (“VEGF”) isoform concentration pattern of the biological material using two or more of a first VEGF cytokine, a second VEGF cytokine, and a third VEGF cytokine;
comparing the VEGF isoform pattern to a reference VEGF isoform concentration pattern that conveys baseline concentration levels of two or more of VEGF165, VEGF121, and VEGF189;
determining a baseline VEGF isoform concentration pattern when the VEGF isoform concentration pattern is within a threshold percentage of the reference VEGF isoform concentration pattern; and
wherein the VEGF isoform concentration pattern conveys concentration levels of two or more of VEGF165, VEGF121, and VEGF189;

13. The method of claim 10, wherein obtaining the biological material comprises:
obtaining a blood sample from the premature infant;
blotting the blood sample on the filter paper; and
drying the blood sample on the filter paper.

14. The method of claim 10, wherein obtaining the biological material comprises obtaining a fecal sample from the premature infant.

15. The method of claim 10, wherein the VEGF isoform concentration pattern is determined using an enzyme-linked immunosorbent assay.

16. The method of claim 13, wherein
the reference VEGF isoform concentration pattern is obtained from premature infants born within a 4-week gestational age range of the first infant.

17. The method of claim 14, wherein the reference VEGF isoform pattern is gender specific.

18. The method of claim 15, further comprising forming the reference VEGF isoform concentration pattern using a statistically significant sampling of premature infants that each exhibit two or more of the following:
a baseline concentration level of VEGF165;
a baseline concentration level of VEGF121; and
a baseline concentration level of VEGF189.
